(19) United States
(12) Patent Application Publication
(10) Pub. No.: US 2026/0224922 A1
(43) Pub. Date: Aug. 6, 2026

(54) HEAT EXCHANGER FOR CLOSED-CIRCUIT REBREATHER

(71) Applicant: CAELI TECHNOLOGIES LTD, Nes Ziyyona (IL)

(72) Inventors: Nir Bloch, Nes Ziyyona (IL); Arie Kalo, Nes Ziyyona (IL); Izhak Rabinovitz, Nes Ziyyona (IL)

(21) Appl. No.: 19/147,171

(22) PCT Filed: Jan. 10, 2024
(86) PCT No.: PCT/IL2024/050036

§ 371 Date: Jul. 10, 2025

(30) Foreign Application Priority Data
Jan. 10, 2023 (IL) 299814

(51) Int. Cl. A62B 7/04; A62B 9/00; A62B 19/00; B01D 53/14; B01D 53/18; B01D 53/62; B01D 53/78

(52) U.S. Cl. CPC A62B 7/04; A62B 9/003; A62B 19/00; B01D 53/1475; B01D 53/18; B01D 53/62; B01D 53/78; B01D 2257/504; B01D 2259/4533; B01D 2259/4541

(57) ABSTRACT

The present invention relates to closed-circuit breathing system with an improved cooling system for prolonged use thereof.

### FIELD OF THE INVENTION

The present invention is in the field of breathing systems, specifically closed-circuit rebreathers for ground use designed for prolonged use.

### BACKGROUND OF THE INVENTION

Self-contained breathing systems are designed to be worn by users in need of oxygen, i.e. people that work in conditions with insufficient oxygen or with hazardous levels of toxic gases, such as rescue workers in hazardous environments, such as smoke-filled or toxic/bad-air environments, in which the atmosphere is unsuitable for breathing. Such systems are classified into closed-circuit and open-circuit systems.

Open-circuit breathing systems are those in which exhaled or expired gases are discharged into the atmosphere and not rebreathed. Although such open-circuit systems are simple and provide excellent protection to the user, the high rate of gas usage and subsequent weight and size of the required gas container, typically limit the usage duration of such systems to no more that about 30 to 45 minutes.

On the other hand, closed-circuit breathing systems, also known as closed-circuit rebreathers (CCRs), provide an extended effective use of about 3 to 4 hours while using a much smaller and lighter gas container (and other components), which is crucial when it is required to work for a long period in such hostile environments, e.g., in underground tunnels, in mine rescue operations, and other confined areas with limited or scarce airflow.

A CCR generally comprises a breathing circuit having an oxygen cylinder with compressed oxygen for supplying oxygen to the breathing circuit, a breathing port, a counterlung (or “breathing bag”), a carbon dioxide (CO₂) absorber, such as a scrubber, and a cooler for cooling the air for inhaling.

When using a CCR, air is inhaled and exhaled through the breathing port, and the gas is continually re-circulated in the closed breathing circuit, while only a small portion of the gas (if any) is released into the atmosphere. When a user inhales and exhales, about 4% of the oxygen within the re-circulated air is consumed and converted into CO₂. Oxygen consumed by the user is replenished by the oxygen supply while CO₂ is removed by the carbon dioxide absorber. The amount of oxygen and carbon dioxide contained in the circulated air is thus maintained at levels that ensure that the user can breathe safely.

It should be appreciated that although the above devices are commonly referred to as “closed-circuit breathing devices”, the breathing circuit is not completely closed. During use, oxygen is added to the breathing circuit and some recirculated air is allowed to vent to the atmosphere. Nevertheless, a substantial proportion of the exhaled air is recirculated within the breathing circuit.

Various CCRs were developed, such as those described in U.S. Pat. No. 4,362,153, U.S. Pat. No. 4,879,996, and U.S. Pat. No. 4,498,470, to provide prolonged breathing time, each one with its disadvantages, such as icing of valves, contamination of air supply by ambient air, overheating of the air within the system and relevant complicated cooling mechanisms, or simply insufficient usage time. For instance, while the CCR of U.S. Pat. No. 4,362,153 enables a relatively prolonged usage/breathing time for a resting user (in which about 7-10 liters of air are consumed per minute), it is designed to provide a constant oxygen pressure that is inadequate for an active user that typically needs more than a 100 liters of air per minute (which results in leakage of ambient air into the user's facepiece). Any attempt to rectify this situation results in the reduction of usage time of the CCR.

The CCR of U.S. Pat. No. 4,879,996 contains a bypass that allows exhaled gas, under strained conditions (i.e., high activity), to bypass the CO₂ scrubber so that untreated exhaled air flows directly back to the user, meaning that CO₂ levels might exceed the allowed maximum level dictated by federal and state regulations (i.e., of 0.5%).

The above and other disadvantages of the prior art CCRs are overcome by the present invention.

### SUMMARY

The present invention provides a closed-circuit rebreather (CCR) (200) comprising:
(a) a dismountable condensed oxygen cylinder (201) designed to provide oxygen; (b) an oxygen regulator (202) attached to said oxygen cylinder (201) and designed to provide oxygen at a predefined pressure; (c) a carbon dioxide (CO₂) absorber (205) comprising one or more dismountable canisters (215) designed to hold a CO₂ scrubber material; (d) a counterlung (206) connected to said CO₂ absorber (205) and designed to receive air exiting therefrom; and (e) a mouthpiece connected to said counterlung (206) via an inlet tube (203) and to said CO₂ absorber (205) via an outlet tube (204), wherein said CCR further comprises a cooling system (300) that comprises a cooling unit (310) comprising: (i) hot air inlets (311) connected to an exit of said CO₂ absorber (205) and designed to receive hot air passing through said CO₂ absorber (205); (ii) cold air outlets (312) designed to pass cold air to said counterlung (206); and a millibar discharge-valve (313) at its bottom section designed to discharge liquid water outside the CCR.

The present invention further provides a closed-circuit rebreather (CCR) (200) comprising: (a) a condensed oxygen cylinder (201); (b) an oxygen regulator (202) attached to said cylinder (201); (c) a counterlung (206); (d) a carbon dioxide (CO₂) absorber (205) comprising one or more dismountable canisters (215) designed to hold a CO₂ scrubber material; (e) a mouthpiece connected to said counterlung (206) via an inlet tube (203) and to said CO₂ absorber (205) via an outlet tube (204); and (f) a cooling system (300) connected to an exit of said CO₂ absorber (205), wherein: said cooling system (300) comprising: (i) a cooling unit (310) attached to said CO₂ absorber (205) for receiving hot air passing therethrough; and (ii) one or more fans or blowers, said cooling unit (310) comprises hot air inlets (311) and cold air outlets (312), and a millibar discharge-valve (313) at its bottom section.

### BRIEF DESCRIPTION OF DRAWINGS

For a better understanding of various embodiments of the invention and to show how the same may be carried into effect, reference will now be made, by way of example, to the accompanying drawings, in which:

FIG. 1 shows a currently known CCR system that is cooled using an ice cube.

FIGS. 2A-2B are illustrations of a CCR system according to an embodiment of the invention: FIG. 2A illustrates the system without a counterlung; and FIG. 2B illustrates the system with a counterlung positioned on top of other components of the system.

FIGS. 3A-3B are illustrations of two possible passages of cooling-air and breathing-air in CCR systems of the invention.

FIGS. 4A-4B illustrate fan units designed to be associated with the cooling system:

FIG. 4A illustrates a two-fan unit; and FIG. 4B illustrates a one-fan unit.

FIGS. 5A-5B are illustrations of two possible cooling mechanisms: FIG. 5A illustrates the use of only ambient air; and FIG. 5B illustrates the use of a refrigerator unit-alone or as an assisting mechanism with ambient air cooling.

FIG. 6 is an illustration of a canister for holding a carbon dioxide scrubber material.

FIGS. 7A-7B are illustrations of a carbon dioxide absorber with 2 canisters holding a carbon dioxide scrubber material: FIG. 7A illustrates the absorber with a cooling system attached thereto, and FIG. 7B illustrates the upper section of the absorber showing the plungers.

FIGS. 8A-8C are illustrations of the mixing of the carbon dioxide scrubber material within the carbon dioxide absorber canisters: FIG. 8A illustrates the airflow directions within the canisters; FIG. 8B illustrates the mixing of the carbon dioxide scrubber material within the canisters; and FIG. 8C illustrates one possible mixer within the canisters that can be used to mix the carbon dioxide scrubber material.

FIGS. 9A-9E illustrate one possible configuration of a cooling unit of the cooling system: FIG. 9A is a front 3D view; FIG. 9B is a rear 3D view; FIG. 9C is a front view; FIG. 9D is an upper view; and FIG. 9E is a bottom view.

FIGS. 10A-10B illustrate two cut sections of the cooling unit of FIG. 9.

FIGS. 11A-11D illustrate an alternative configuration of the cooling unit of the cooling system, having a lattice construct: FIG. 11A is a front 3D view; FIG. 11B is a front-lower 3D; FIG. 11C is an upper 3D view; and FIG. 11D is a lower 3D view.

FIGS. 12A-12B illustrate two cut-section views of another alternative configuration of the cooling unit of the cooling system, having an internal lattice construct.

Structural details of the invention are shown to provide a fundamental understanding of the invention, the description, taken with the drawings, making apparent to those skilled in the art how the several forms of the invention may be embodied in practice.

### DETAILED DESCRIPTION

Emergency rescue personnel, tunnel fighting soldiers, and other workers and personnel that are required to operate under oxygen-depleted or air-contaminated environments, such as underground tunnels, collapsed mines and tunnels, high mountains (with “thin-air”), toxic environments (for example, near a toxic waste plant), etc., must use an air supply system to provide them with oxygen.

The problem is that due to the size and weight of standard systems, they can be used for only a short amount of time before oxygen runs out. To increase usage of such a system and prolong the user's activity time, closed-circuit rebreathers (CCRs) were developed, in which exhaled air is reused after carbon dioxide (CO₂) is removed. Such systems enable the user to dramatically extend his/her activity time while using the same sized air tank as used in the open-circuit breathing systems.

However, the removal of the CO₂ presented another issue: heat. Removal of CO₂ in the carbon dioxide absorber of such CCRs is done using various known chemicals, such as zeolites and soda lime. Absorbance of CO₂ by such chemicals is carried out by an exothermic chemical reaction, which causes the air passing through the carbon dioxide absorber to reach high temperatures. Since breathing hot air is not recommended, such CCRs had to use a cooling system or mechanism to cool the air back to an allowed temperature before it is inhaled by the user.

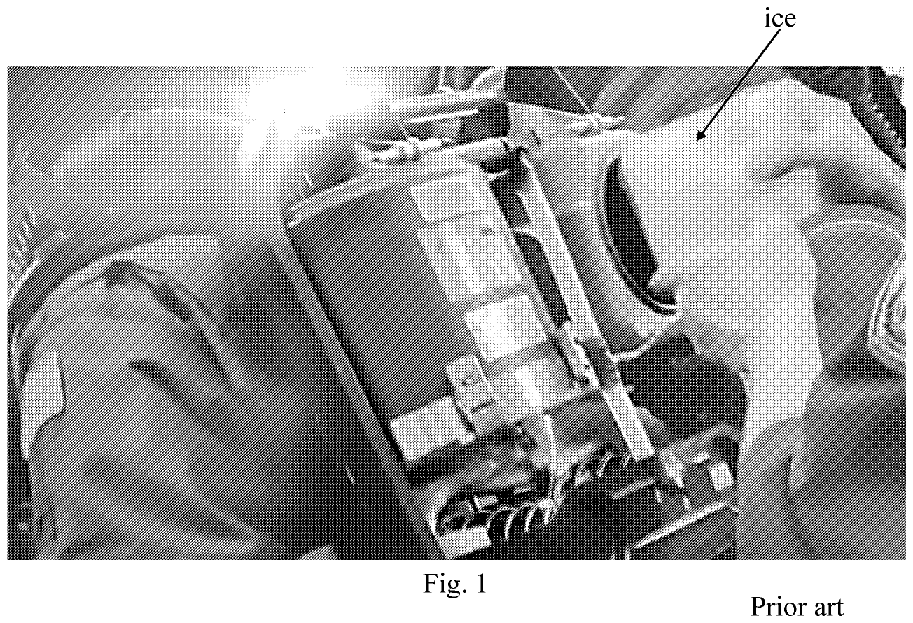

Currently, the cooling of such CCRs is done by placing ice cubes inside the system (as done in Drager's rebreather-see FIG. 1). However, this cooling technique is very limiting—it is effective only while the ice lasts, and it requires the user to constantly replace the ice cube, as well as maintain an ice reservoir.

Accordingly, the present invention provides CCRs with novel and improved cooling systems that maintain the air in the system intended for breathing cool, namely at a desirable temperature that is suitable for breathing, while enabling prolonged usage time of the system without refilling or replacing system's components, such as the oxygen tank, the cooling element, batteries, and the carbon dioxide absorber.

Thus, in a first aspect, the present invention provides a closed-circuit rebreather (CCR) (200) comprising: (a) a dismountable condensed oxygen cylinder (201) designed to provide oxygen; (b) an oxygen regulator (202) attached to said oxygen cylinder (201) and designed to provide oxygen at a predefined pressure; (c) a carbon dioxide (CO₂) absorber (205) comprising one or more dismountable canisters (215) designed to hold a CO₂ scrubber material; (d) a counterlung (206) connected to said CO₂ absorber (205) and designed to receive air exiting therefrom; and (e) a mouthpiece connected to said counterlung (206) via an inlet tube (203) and to said CO₂ absorber (205) via an outlet tube (204), wherein said CCR further comprises a (electric) cooling system (300) that comprises a cooling unit (310) comprising: (i) hot air inlets (311) connected to an exit of said CO₂ absorber (205) and designed to receive hot air passing through said CO₂ absorber (205); (ii) cold air outlets (312) designed to pass cold air to said counterlung (206); and a millibar discharge-valve (313) at its bottom section designed to discharge liquid water outside the CCR.

In certain embodiments, the present invention provides a closed-circuit rebreather (CCR) (200) comprising: (a) a condensed oxygen tank/cylinder (201) for supplying oxygen to the user of the CCR; (b) an oxygen regulator (202) attached to the tank (201) to regulate oxygen flow and pressure within the system; (c) a counterlung (or breathing bag) (206); (d) a carbon dioxide (CO₂) absorber/scrubber (205) comprising one or more dismountable canisters (215) designed to hold a CO₂ scrubber material for removal of CO₂; (e) a gas breathing port/mouthpiece connected to the counterlung (206) via an inlet tube (203) and to the CO₂ absorber (205) via an outlet tube (204); and (f) a (electric) cooling system (300) connected to an exit of the CO₂ absorber (205) for cooling air passing through the CO₂ absorber (205) and to the counterlung (206) for delivering the air that was cooled thereby, wherein the cooling system (300) comprises: (i) a cooling unit (310) attached to the CO₂ absorber (205) for receiving hot air passing therethrough; and (ii) one or more fans or blowers, and wherein the cooling unit (310) comprises hot air inlets (311) and cold air outlets (312), and a millibar discharge-valve (313) at its bottom section.

Notably, the CCR (200) of the invention is designed to be used by a user on land. As such, to simplify the use thereof, the CCR may be equipped with shoulder straps to enable the user to wear the CCR on his/her back while keeping his/her hands free. In specific embodiments, the CCR is placed in, or is an integral part of, a backpack having shoulder straps. In specific embodiments, the backpack is, or comprises, an inner backpack panel (400) to which the CCR is anchored/mounted on.

In the CCR of the invention, oxygen exits the oxygen cylinder (201) and flows to the mouthpiece via an inlet tube (203). After the user inhales the oxygen, he/she exhales a mixture of oxygen and carbon dioxide (CO₂) that flows through an outlet tube (204) to the CO₂absorber (205). As the mixture of oxygen and carbon dioxide flows through the CO₂ absorber (205), CO₂ is removed via an exothermic chemical reaction with the CO₂ scrubber material. The air that exits the CO₂ absorber (205) is thus relatively hot (about 50° C.). In the CCR of the invention, the hot air exits the CO₂ absorber (205) and enters the cooling unit (310) via dedicated inlets (311) that direct the hot air into multiple passageways/ducts/funnels for cooling thereof. The cooling unit (310) acts as a radiator to cool the air within. In specific embodiments, this is done by passing ambient air through the external ribs of the cooling unit (310) and between the passageways/ducts/funnels. In alternative or added embodiments, this is done by using a refrigerator unit that either operates alone or in combination with the passing of the ambient air. Once the air exits the cooling unit (310), it enters into the counterlung (206), before returning to the user. Additional oxygen is added to the air exiting the counterlung from the oxygen cylinder (201) according to need to bring the oxygen pressure reaching the mouthpiece to a desired level.

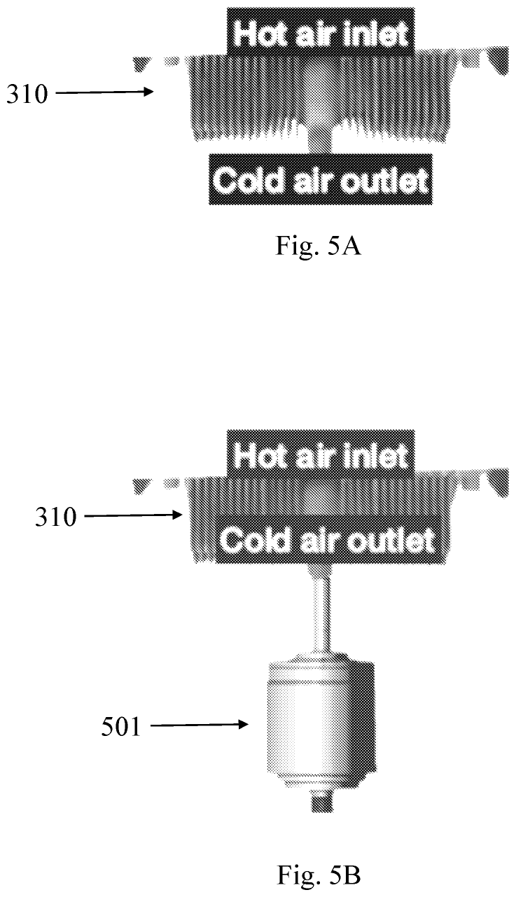

As explained above, the hot air exiting the CO₂ absorber (205) enters the cooling unit for cooling. As such, the cooling system of the CCR according to any of the embodiments above may include one or more fans or blowers designed to create airflow over said cooling unit (310) for cooling the hot air passing therethrough. Alternatively, or in addition to such fans, the cooling system may further comprise an electric cryocooler (illustrated in FIG. 5B) or a liquid-based cooling cycle holding a liquid coolant, said cooling cycle comprises an evaporator and a condenser, and optionally a pressure-or vacuum-generator. This option is especially important when the ambient temperature is higher than the temperature of the hot air exiting the CO₂ absorber (205), in which case simply blowing ambient air over the cooling unit would not be sufficient to cool the hot air inside it to the desired temperature. Notably, such electric cryocooler or liquid-based cooling cycle can be used with or without said fans or blowers. For instance, when the CCR includes both fans and electric cryocooler, to save power, the cryocooler may be activated only when needed, i.e., when the ambient temperature exceeds a certain temperature. Alternatively, the CCR may include only one or more fans or only electric cryocooler.

Since the CO₂ absorber (205) emits heat, it might be advisable, in certain cases, to separate it from other components of the CCR, such as from the counterlung that might be damaged due to excessive heat. Accordingly, in certain embodiments, the CCR according to any of the embodiments above further comprises a heatshield cover (207) positioned between said counterlung (206) and said CO₂ absorber (205). Alternatively, the CCR further comprises a container (instead of the heatshield cover) holding said CO₂ absorber (205). In specific embodiments, in either case, i.e., when there is a heatshield cover (207) or a container, the cooling system (300) is designed to direct airflow from, e.g., said one or more fans or blowers over said cooling unit (310) and over said CO₂ absorber (205) for cooling thereof.

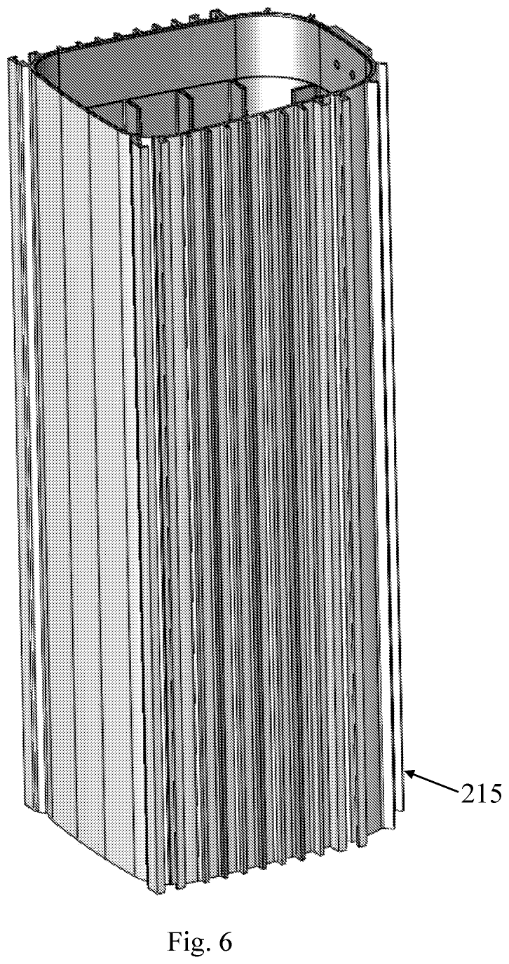

Notably, and as illustrated in FIG. 6, the canisters holding the CO₂ absorber (205) are designed such that they have a large area surface to improve heat dispersion and cooling thereof. Accordingly, in certain embodiments of the CCR according to any of the embodiments above, the CO₂ absorber (205) comprises two or more (dismountable) canisters (215) designed to hold a CO₂ scrubber material. In specific embodiments, the canisters (215) include external cooling ribs and internal ribs to increase overall surface to improve heat transfer.

Notably, when oxygen is inhaled, it absorbs moisture from the user's respiratory system. As a result, the air exhaled from the user's lungs contains water molecules/vapors that, during the cooling of the air in the cooling unit (310), are condensed into liquid water. The liquid water is then removed from the CCR system of the invention via the millibar discharge-valve (313) at the cooling unit's (310) bottom section, thereby removing any risks associated with the presence of liquid in the CCR. Notably, the CCR (200) according to any of the embodiments above is designed to be carried on the user's back, which means that the CCR is in an upright position with a “top/upper” and a “bottom/lower” orientations, such that the “bottom” of the CCR is the lowest point when the user is standing up. Accordingly, when the user is standing, the millibar discharge-valve (313) is positioned at the bottom section of the cooling unit (310), which allows condensed water to flow outwardly. In alternative or additional embodiments, the CCR (200) further comprises a pump, plunger, or any other suitable means designed to push such condensed water out from said millibar discharge-valve (313), even when the user is in a horizontal position, e.g., when crawling in a narrow cave, which also positions the CCR in a horizontal position so that the millibar discharge-valve (313) is no longer at the “bottom”.

In certain embodiments, the CCR (200) comprises an additional oxygen regulator located-at or associated-with the mouthpiece to provide a desired amount of oxygen to the user according to need. For instance, under standard working conditions, when the user needs more oxygen, higher oxygen pressure is generated to deliver more oxygen to the user, and vise-versa.

One of the problems of current CCRs is that they do not enable the user to replace disposable components in a fast and simple manner, which means that once replacement is needed, e.g., of the oxygen cylinder or the CO₂ absorber (205) or the CO₂ scrubber material or the power source, the user is forced to stop working and exit the working area to get assistance in replacing what is needed to be replaced.

Contrary to that, the present CCR (200) enables fast and simple replacement of each and every part/component within the CCR or replacement of the entire CCR unit as a whole. Accordingly, the present CCR according to any of the embodiments above comprises quick-release latches designed to enable quick and simple release of the different components within the CCR. Alternatively, the CCR comprises quick-release latches designed to enable quick and simple release of the entire CCR unit from its mount carried by the user. This means that the CCR can be quickly and easily disassembled and reassembled by the user himself in the field without needing to stop working or by stopping for a very short time. For instance, the user can take a deep breath and hold it for about 30 seconds (if needed), during which he/she can replace (optionally by the aid of a partner) any desired component or the entire CCR.

In certain embodiments, as mentioned above, the CCR (200) according to any of the embodiments above further comprises a container in which the CO₂ absorber (205) and the cooling system (300) reside. Alternatively, the CCR comprises a heatshield cover (207) separating the CO₂ absorber (205) from other components of the CCR, such as the counterlung. This container or heatshield are designed to direct the airflow from the one or more fans or blowers over the cooling unit (310) and then over the CO₂ absorber (205). In this way, (ambient or chilled) air blown by the fans or blowers is forced to pass through the cooling unit and over the CO₂ absorber, for cooling thereof, and is not dispersed randomly within the CCR. Moreover, the fact that the air passes over the CO₂ absorber (205) enables it to cool the CO₂ absorber itself and thus reduce the overall temperature of the hot air exiting therefrom, thereby assisting in the cooling of the air by the cooling unit (300) itself.

Another advantage that the above container or heatshield provide, is the separation of the CO₂ absorber (205) (and optionally also the cooling system (300)) from the counterlung (206), which reduces the risk of unintentional damage to the counterlung (206) due to excessive heat generated by the CO₂ absorber (205).

The removal of CO₂ from the exhaled air is carried out inside the CO₂ absorber (205). Accordingly, the more CO₂ scrubber material is present in the CCR, the more CO₂ is removed and the longer the user can use the CCR before needing to replace the CO₂ scrubber material or the CO₂ absorber (205). Accordingly, in specific embodiments of the CCR (200) according to any of the embodiments above, the CO₂ absorber (205) comprises two or more dismountable canisters (215) designed to hold a CO₂ scrubber material. In further specific embodiments of the CCR (200) according to any of the embodiments above, the CO₂ absorber (205) comprises two such dismountable canisters (215).

Notably, the CO₂ scrubber material residing within the canisters (215) may shift and move within the canister thereby creating undesired passageways for exhaled air to pass through quickly without actually passing through the CO₂ scrubber material itself, which means that no or little CO₂ will be removed. To avoid this issue, it is important to maintain the CO₂ scrubber material condensed at all times and avoid movement thereof. Accordingly, in certain embodiments of the CCR (200) according to any of the embodiments above, the CO₂ absorber (205) comprises or is equipped with an upper lid having a plunger (225) for each canister (215) therein, the plunger (225) is designed to press the CO₂ scrubber material within said canister(s) (215) and maintain it under constant pressure at all times, regardless of the position of the CCR or the effects of external forces applied thereon (shaking, etc.) such that no passageways or cracks are formed within the CO₂ scrubber material. This may assist in maintaining the efficiency of the CCR during prolonged storage and during transport, which may lead to the creation of such passageways or cracks in the CO₂ scrubber material inside the canisters.

In specific embodiments, the present invention provides a closed-circuit rebreather (CCR) (200) comprising: (a) a dismountable condensed oxygen cylinder (201) designed to provide oxygen; (b) an oxygen regulator (202) attached to said oxygen cylinder (201) and designed to provide oxygen at a predefined pressure; (c) a carbon dioxide (CO₂) absorber (205) comprising one or more dismountable canisters (215) designed to hold a CO₂ scrubber material; (d) a heatshield cover (207); (e) a counterlung (206) connected to said CO₂absorber (205) and designed to receive air exiting therefrom; (e) a mouthpiece connected to said counterlung (206) via an inlet tube (203) and to said CO₂ absorber (205) via an outlet tube (204); and (f) an electric cooling system (300) that comprises a cooling unit (310) comprising: (i) hot air inlets (311) connected to an exit of said CO₂ absorber (205) and designed to receive hot air passing through said CO₂ absorber (205); (ii) cold air outlets (312) designed to pass cold air to said counterlung (206); (iii) a millibar discharge-valve (313) at its bottom section designed to discharge liquid water outside the CCR; and (iv) one or more fans or blowers and/or an electric cryocooler, wherein said heatshield cover (207) is designed to direct airflow from said one or more fans or blowers over said cooling unit (310) and over said CO₂ absorber (205).

In further specific embodiments, the CO₂ absorber (205) comprises: (i) an upper lid with a plunger (225) designed to press the CO₂ scrubber material within each one of said one or more canisters (215) and maintain it under constant pressure at all times; and/or (ii) a mixer for mixing the CO₂ scrubber material within each one of said one or more canisters (215). In yet further specific embodiments, each one of said two or more canisters (215) contain CO₂ scrubber material.

In specific embodiments, the present invention provides a closed-circuit rebreather (CCR) (200) comprising: (a) a condensed oxygen cylinder (201); (b) an oxygen regulator (202) attached to said cylinder (201); (c) a container holding a CO₂ absorber (205) and an electric cooling system (300) connected to an exit of said CO₂ absorber (205), said CO₂ absorber (205) comprises two or more dismountable canisters (215) designed to hold a CO₂scrubber material; (d) a counterlung (206); and (e) a mouthpiece connected to said counterlung (206) via an inlet tube (203) and to said CO₂ absorber (205) via an outlet tube (204); wherein: (i) said cooling system (300) comprises: (i) a cooling unit (310) that comprises hot air inlets (311) and cold air outlets (312), and a millibar discharge-valve (313) at its bottom section, wherein said cooling system (300) is attached to said CO₂ absorber (205) for receiving hot air passing therethrough; and (ii) one or more fans or blowers and/or an electric cryocooler; wherein said container is designed to direct airflow from said one or more fans or blowers over said cooling unit (310) and over said CO₂ absorber (205).

In further specific embodiments, the CO₂ absorber (205) comprises: (i) an upper lid with a plunger (225) designed to press the CO₂ scrubber material within each one of said one or more canisters (215) and maintain it under constant pressure at all times; and/or (ii) a mixer for mixing the CO₂ scrubber material within each one of said one or more canisters (215). In yet further specific embodiments, each one of said two or more canisters (215) contain CO₂ scrubber material.

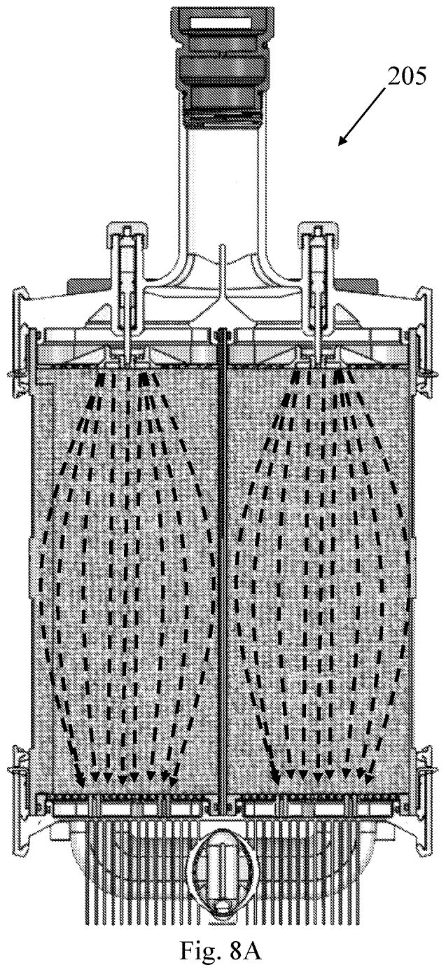

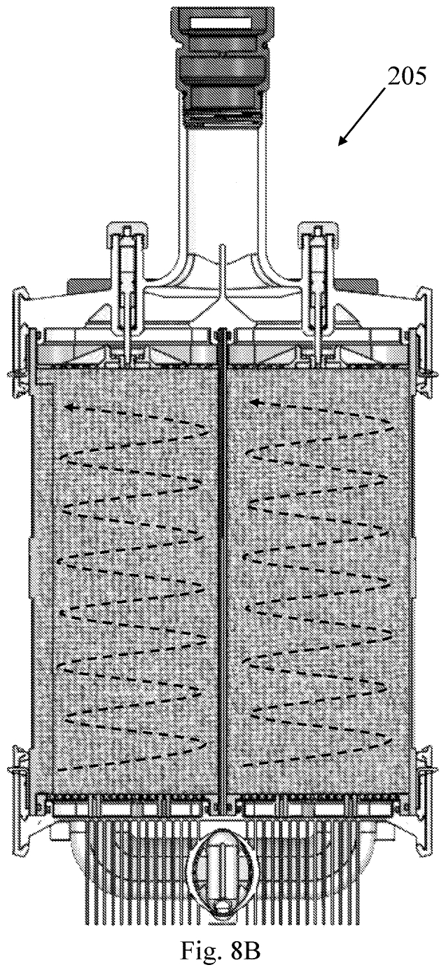

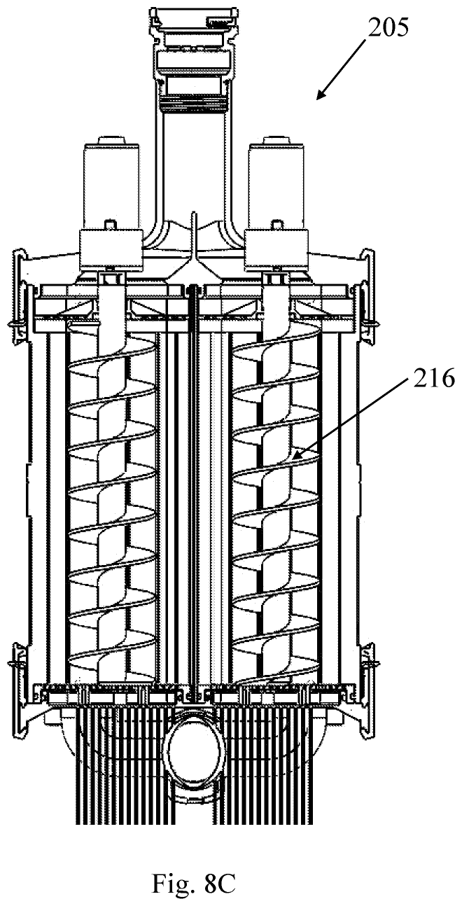

The term “mixer” as used herein may be either a physical mixer, such as an auger-like (216) rod (see illustrated in FIG. 8C), in which case the rotation movement of the rod forces the mixing of the scrubber material within the canister; or is based on airflow within the canister, e.g., using dedicated pipes and air pumps, in which case the passage of air within the canister forces the mixing of the scrubber material. In specific embodiments, the air passing through the scrubber material in the canister passes through the cooling unit and thus aids in the cooling of the CO₂ absorber (205).

As explained above, exhaled air contains water vapors. Accordingly, the present CCR (200) comprises a millibar discharge-valve (313) at the cooling unit (310) for discharging any condensed liquid generated during the cooling process. However, some moisture or water vapors may remain in the air and may condense during passage within the inlet tube (203) leading air from the counterlung (206) to the mouthpiece. Accordingly, in certain embodiments of the CCR (200) according to any of the embodiments above, the mouthpiece further comprises an additional liquid discharge-valve for discharging liquids (water) accumulated in the system during use thereof.

In certain embodiments of the CCR according to any of the embodiments above, all the components of the CCR are dismantlable via quick-release latches. In alternative or added embodiments, the entire CCR unit is dismantlable via quick-release latches to a backpack carried by the user, thereby enabling quick and simple release and replacement of the entire CCR.

In a second aspect, the present invention provides a closed-circuit breathing rebreather (CCR) (200) as defined above, the improvement thereof is the presence of an electric cooling system (300). In specific embodiments, the cooling system comprises a cooling unit (310) having a millibar discharge-valve (313) at its bottom section for discharging liquids accumulated in the system during use thereof. The electric cooling system (300) enables cooling the air within the CCR to a desired temperature that is even lower than ambient temperature.

In certain embodiments, the above CCR (200) further comprises an additional discharge-valve at the mouthpiece.

In a third aspect, the present invention provides a method for enabling a user of a CCR system to use such CCR system for a prolonged period under conditions that require the use of a CCR system, such as a high-temperature environment, wherein the method is carried out using the CCR system according to any of the embodiments above. Accordingly, the method of the invention comprises the steps of providing the system according to any of the embodiments above and activating same for activating the cooling system to chill the air exiting the CO₂ scrubber and before it enters the counterlung. Once activated, the cooling unit passes ambient or chilled air over the cooling unit, or directly chills the cooling unit, thereby cooling the hot air passing therethrough before it exits and enters the counterlung.

The invention will now be illustrated by reference to the accompanying drawings which are to be considered only as representative examples of possible embodiments of packages of the invention. Alterations and modifications may be made by those having ordinary skill in the art without departing from the scope of the invention. Therefore, it must be understood that the illustrated embodiment has been set forth only for the purpose of example and that it should not be taken as limiting the invention as defined by the following invention and its various embodiments and/or by the following claims. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

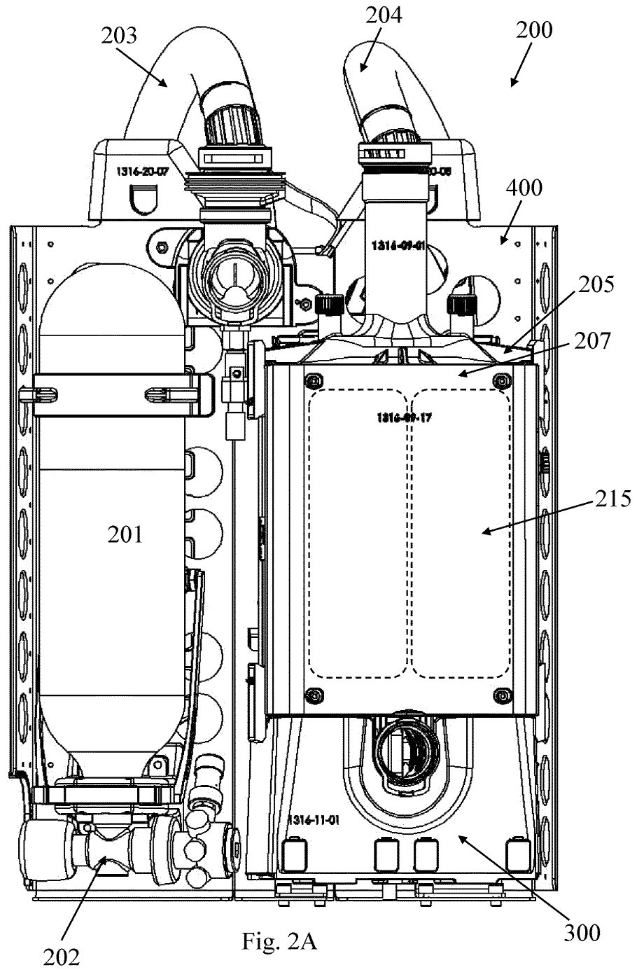

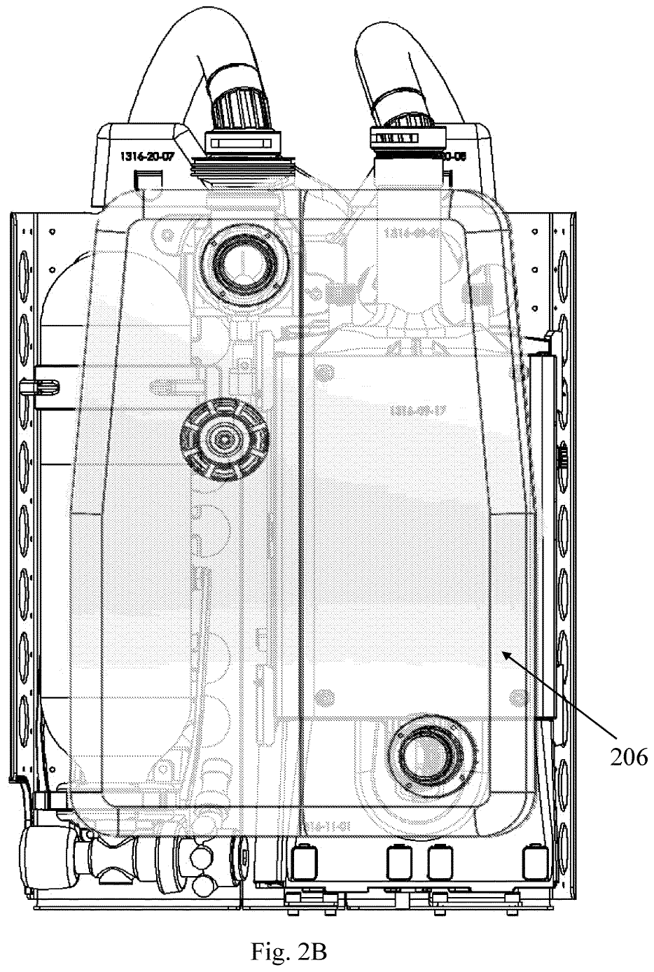

FIG. 2 illustrates a closed-circuit rebreather (CCR) (200) according to one embodiment of the invention, illustrating the different main components thereof: a condensed oxygen tank/cylinder (201) for supplying oxygen; an oxygen cylinder's regulator (202) attached to the cylinder (201) to regulate oxygen flow and pressure within the rebreather; a carbon dioxide (CO₂) absorber/scrubber (205) for removal of CO₂ from breathed air; a counterlung (206) (or breathing bag) receiving CO₂ -deprived air exiting from the CO₂ absorber/scrubber (205) and having an exit, optionally with another regulator, to a breathing/inhalation hose (203) for delivering oxygen to the user; an exhalation breathing hose (204) delivering exhaled gas from the user to the CO₂ absorber (205); and a cooling system (300) connected to an exit of the CO₂ absorber (205) for cooling air passing through the CO₂ absorber (205), and then passing the cooled air into the counterlung (206). The cooling system (300) comprises a cooling unit (310) through which hot air from the CO₂ absorber passes and one or more cooling fans for passing ambient air over the exterior of the cooling unit (310) to cool the hot air inside it.

Also illustrated in FIG. 2 is a rigid backpack-panel/base (400) made of a rigid material, such as plastic, aluminum, metal, polycarbonate, etc., onto which the different components of the CCR (or the entire CCR as a whole) are mounted. The assembly of the CCR components onto the backpack-panel (400) is based on quick-release latches that facilitate fast replacement thereof when needed and without complicated and cumbersome replacement of parts as done today in known CCRs.

In certain embodiments, the backpack-panel (400) with all the CCR components thereon may be attached to a backpack using quick-release latches (not shown) such that a user carrying the backpack can replace the entire CCR unit quickly and easily without needing to remove the backpack from his/her back.

The CO₂ absorber (205) is designed to hold CO₂ scrubber material. The material can be in any form and constellation, such as free powder, sacks holding the powder, a liquid or a gel, or any other. The material is placed in a dedicated canister (215), such as that illustrated in FIG. 6. The CO₂ absorber (205) may include 1, 2, 3, 4, 5, 6, or more such canisters (215), which can be in any size and shape according to the overall structure, size, and shape of the CO₂ absorber (205). As illustrated in FIG. 6, the canister (215) may have inner protrusions designed to provide support for the CO₂ scrubber material placed within, as well as assist in heat removal by transferring heat from within the canister (215) towards its external surface. In addition, the canister (215) may have external protrusions (or cooling ribs) designed to assist in heat transfer/dissipation/removal by increasing the surface area of the canister (215). FIG. 2B illustrates the same CCR unit of FIG. 1A, only with the counterlung placed over the different components. As can be seen, the counterlung is separated from the CO₂ absorber (205) using a heatshield that can be a simple cover (207) placed over the CO₂ absorber (205) or a container holding the CO₂ absorber (205) and optionally the cooling system. the purpose of the heatshield cover (207) or container, in addition to the protection of the counterlung from heat radiating from the CO₂ absorber (205), is to also direct airflow from the cooling system over the CO₂ absorber (205) to improve cooling efficiency.

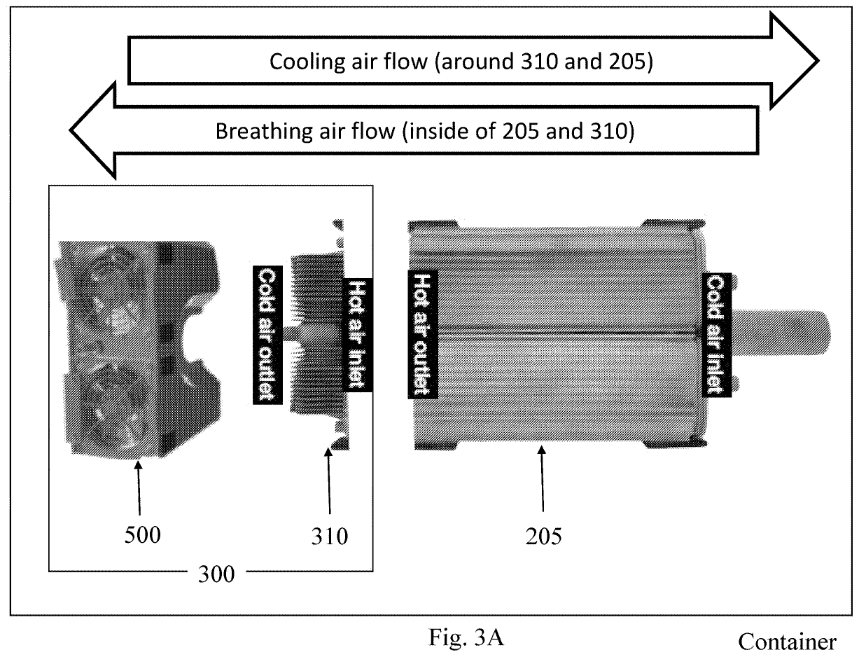

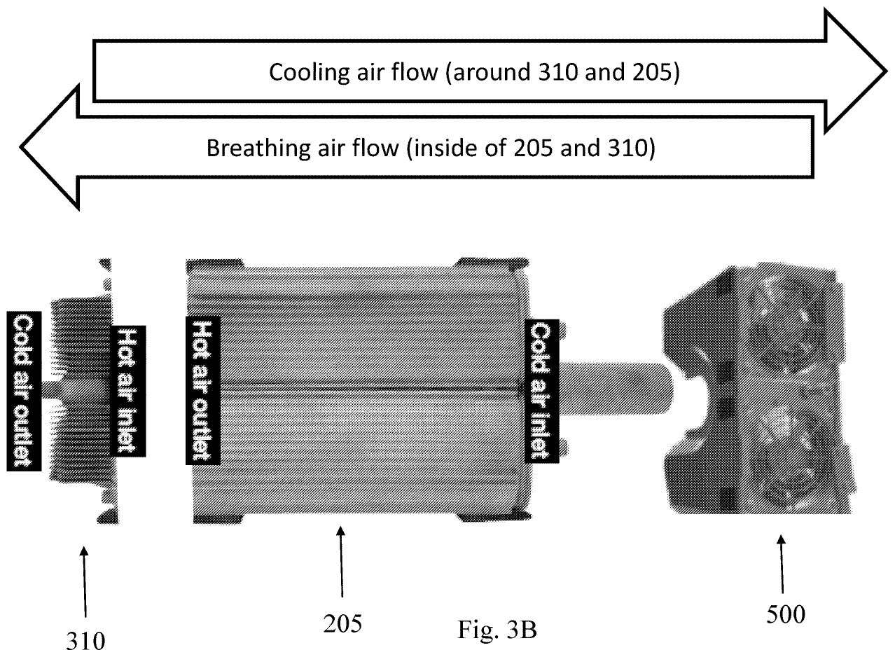

FIG. 3A illustrates one possible configuration of the fans component of the cooling system (300) in which the fans (500) are positioned near the cold air outlet of the cooling unit (310): as illustrated, breathed air enters the CO₂ absorber (205) and CO₂ -depleted hot air exits the CO₂ absorber (205) into the cooling unit (310) onto which (cold) air is blowen using fans (500). The blown air cools the hot air, which then exits the cooling unit (310) and enters the counterlung.

FIG. 3B illustrates an alternative possible configuration of the fans component of the cooling system (300) in which the fans (500) are positioned near the cold air inlet of the CO₂ absorber (205): as illustrated, breathed air enters the CO₂ absorber (205) and CO₂ -depleted hot air exits the CO₂ absorber (205) into the cooling unit (310) onto which (cold) air is passed using fans (500) that draw air “upwardly”. The drawn air cools the hot air, which then exits the cooling unit (310) and enters the counterlung.

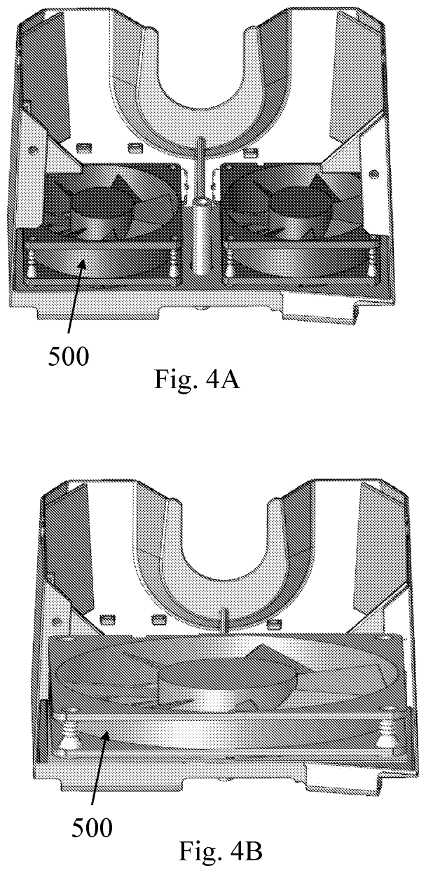

Notably, the location of the fans (500) in the CCR can vary as long as they enable or facilitate airflow passage over the cooling unit (310). For instance, fans can be positioned both near the cold air outlet of the cooling unit (310) and near the cold air inlet of the CO₂ absorber (205) (not shown). In addition, the number of fans (500) being used can vary, and can be 1, 2, 3, 4, or more fans, according to need, desire, and design. FIG. 4A illustrates a 2-fan design, and FIG. 4B illustrates a single-fan design.

It should also be noted that in addition to, or instead of, the fans illustrated in FIGS. 3A and 3B, the cooling system (300) may include, e.g., a refrigerator unit or cryocooler that either replaces the fans or assists the fans, e.g., when the ambient temperature is higher that the desired temperature of the air for breathing. FIG. 5A illustrates one possible heatsink version of the cooling system, in which only ambient air is used to cool the air, and FIG. 5B illustrates an alternative possible heatsink version of the cooling system, in which an assistive refrigerator unit (501) or cryocooler is used-either as a sole cooling mechanism or in conjunction with fans that passes ambient air. The use of such refrigerator unit (501) or cryocooler can be for cooling the ambient air before passing it over the cooling unit (310) or for directly cooling the cooling unit (310) itself, or both.

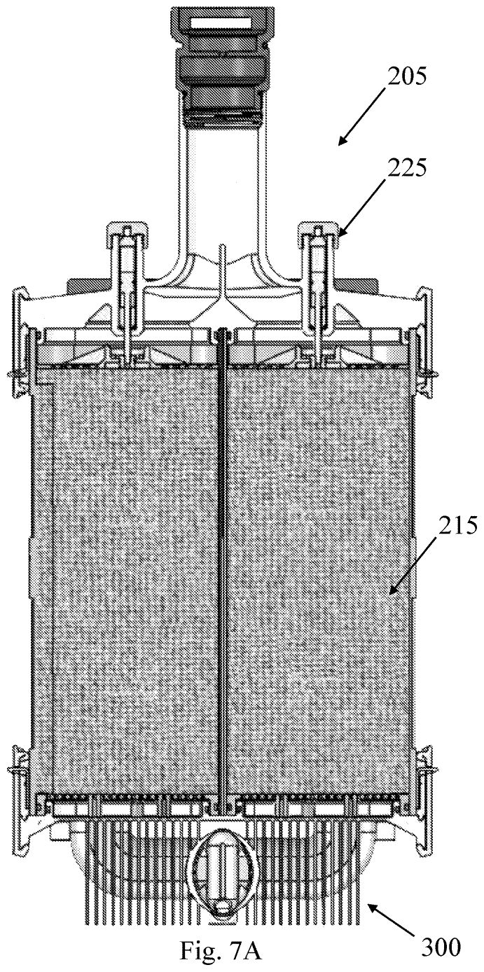

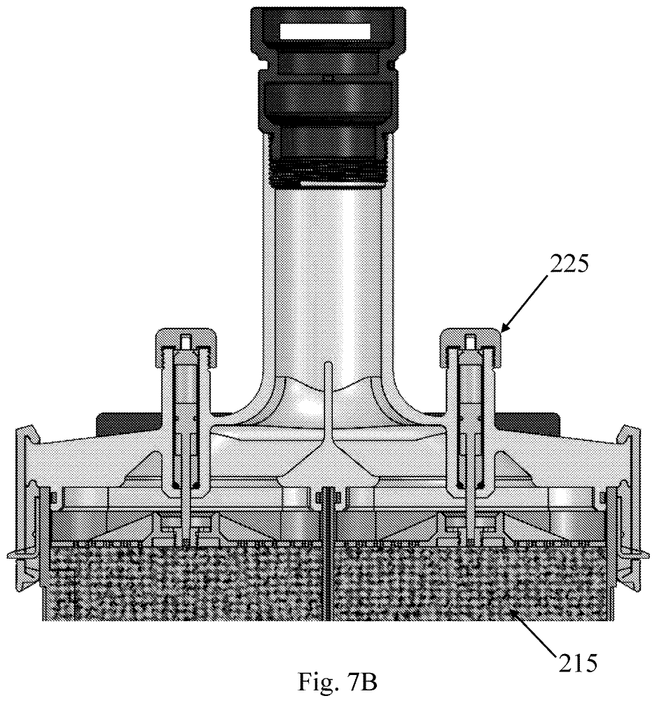

FIG. 7A illustrates a CO₂ -absorber (205) according to an embodiment of the invention having two canisters (215) holding the CO₂ scrubber material. In the illustrated design, exhaled air is delivered to the CO₂-absorber (205) where it splits into two routes, each leading to one of the canisters (215), and then the CO₂ -depleted air exits the canisters (215) and enters the cooling system (300).

As noted above, the CO₂ scrubber material may be in the form of a powder. This may present a problem due to storage and transport conditions: when placing a powder in a canister, and then placing the canister in a horizontal position, the powder may be condensed against the side walls of the canister thereby creating “passageways” or “cracks” within the material in the canister that do not comprise active material, which means that when air passes through the canister it would not go through the CO₂ scrubber material and CO₂ will not be removed. This problem may increase when the absorber (205) is in a laying position and is shaken (e.g., during transport or activity of the user). Accordingly, as illustrated in FIGS. 7A and 7B, the present invention further provides a CO₂ absorber (205) with a dedicated plunger (225) for each canister (215). Such plunger (225) is designed to maintain the CO₂ scrubber material within the canister under constant pressure at all times, such that no “passageways”/“cracks” are formed regardless of the position and condition of the absorber (205). Notably, although FIG. 7A illustrates that the plungers (225) are positioned at the upper section of the canisters (215), they can be positioned at the bottom section of the canisters (not shown).

In certain embodiments of the CCR (200) according to any of the embodiments above, each one of the one or more canisters (215) contain CO₂ scrubber material.

In certain embodiments, the CO₂ absorber (205) comprises dismantlable parts that can be disassembled and reassembled according to need. For instance, when replacing damaged parts or when needing to replace the CO₂ scrubber material or the CO₂ absorber (205) itself. In specific embodiments, the CO₂ absorber (205) comprises 2 canisters (215); an upper lid with a plunger (225) for each canister (215); and a bottom lid that is part-of or is associated-with the cooling unit (310). The assembly and disassembly of the different components is carried out using quick-release latches (a.k.a. draw latches).

In specific embodiments, the canister (215) is made of a thin metal having a high heat transfer coefficient. As illustrated in FIG. 6, the canister (215) may further include external cooling ribs and internal ribs to increase overall surface to improve heat transfer, such that heat generated within the canister due to CO₂ removal is partially removed through the canister's walls/surface thereby assisting in the cooling of the air that exits the CO₂ absorber (205). Notably, the inner ribs assist in transferring heat from within the canister (215) to the external outer surface thereof.

When air passes through the CO₂ absorber (205), CO₂ molecules are removed from the air by reacting with the CO₂ scrubber material. Such a reaction actually neutralizes the CO₂ scrubber material that was involved in the reaction, which makes it ineffective when additional air passes therethrough. Eventually, the majority of the CO₂ scrubber material is used, and the user needs to replace the CO₂ scrubber material or the canisters holding the material (or the entire CCR). Optimally, the amount of CO₂ scrubber material used in a CCR is measured according to the amount of oxygen in the oxygen cylinder, such that when the cylinder is depleted, also the CO₂ scrubber material needs changing. However, due to the structure of the CO₂ absorber (205) and the canisters (215) holding the CO₂ scrubber material, and the direction and flow-orientation of the air passing within the canisters, it has been found that a large amount of the CO₂ scrubber material is not used, which means that current CCR systems use excessive amount of CO₂ scrubber material, which increase the weight of the system. This is illustrated in FIG. 8A which shows that air passes (dashed lines) mainly at the center of the canister, and does not react with CO₂ scrubber material at the canister's corners and sides.

Accordingly, in certain embodiments of the CCR (200) according to any of the embodiments above, the CO₂ absorber (205) or canister(s) (215) further comprise a mixer (216) for mixing the CO₂ scrubber material within the canisters (215). Such a mixer (216) causes the CO₂ scrubber material to mix in the canister (see illustrated in FIG. 8B) thereby bringing “unused” material from remote, unreached corners of the canister into the passageway of the air's direction and flow-orientation as it passes within the canisters. In this way, it is possible to reduce the amount of CO₂ scrubber material that needs to be used in current CCRs and/or to extend the usage time of the CCR before needing to replace the CO₂absorber (205).

The mixer can be either a mechanical element within the canister that physically moves the CO₂ scrubber material. For instance, FIG. 8C illustrates a screw-like mechanism (216) located within the canister, such that its rotations cause movement and mixing of the CO₂scrubber material in the canister. Other mechanic mechanisms can be used as well, such as rods moving up and down in the canister, rotating rods, or even a stirrer that is rotated within the canister using a magnet.

Alternatively, the mixer can be a non-physical element, meaning that air or oxygen is used to mix the CO₂ scrubber within the canister (not shown). This can be done by using fans or blowers associated with the canister, e.g., as part of the lid or bottom, that blows air into the canister and causes turbulence of the material within it. Alternatively, one can air outlets designed to blow oxygen (from the oxygen canister (201)) into the canister, such that the pressured oxygen entering the canister creates turbulence of the CO₂ scrubber material.

In such cases, when the CCR (200) comprises such a mixer (216), it should be noted that the mixer (216) is active interchangeably and not constantly. As such, before the mixer is activated, the plunger(s) (225), if present, are released to remove the pressure onto the CO₂ scrubber material within the canister (215) thereby enabling its mixing. Once the mixing is complete (within a few seconds), the plunger(s) (225) are reactivated, and apply pressure onto the CO₂ scrubber material to, as explained above, prevent the formation/creation of undesired passageways within the CO₂ scrubber material for exhaled air to pass through quickly without passing through the CO₂ scrubber material itself.

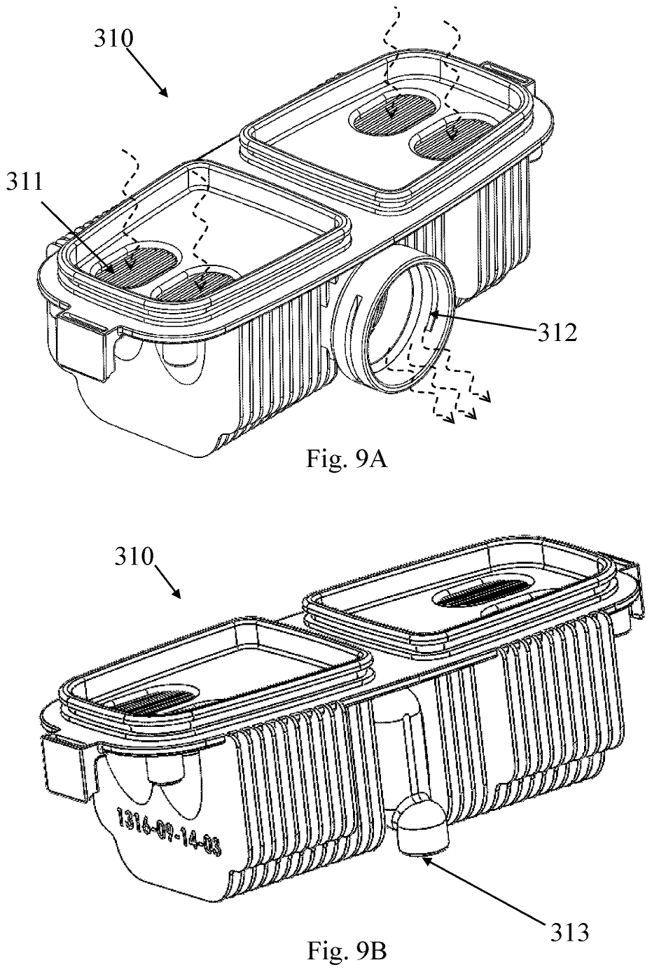

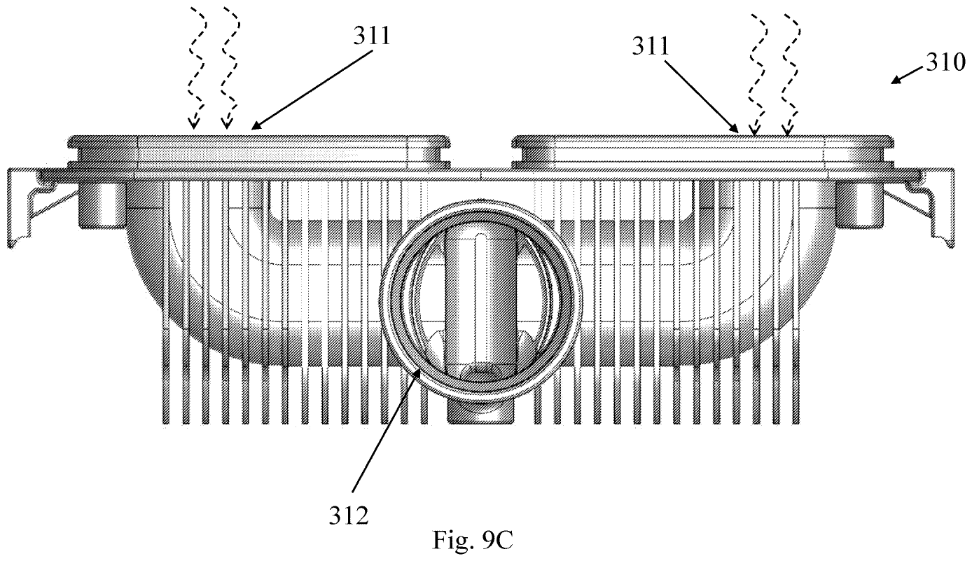

FIGS. 9A-9E illustrate one possible configuration of a cooling unit (310) according to some embodiments of the invention. As illustrated, the cooling unit (310) is designed to fit a two-canister CO₂ absorber: hot air exiting each canister enters the air inlets (311), passes through the unit (310) for cooling thereof, and exits through a single air outlet (312) that connects the cooling unit (310) to the counterlung (206).

The cooling system (300) is a heat transfer system that is based on heat convection and is designed to exchange heat between the hot air passing through the cooling unit (310) and ambient air that passes externally thereto. In certain embodiments, the cooling system (300) acts as a radiator or heat sink to cool down the hot air after it exits the CO₂ absorber (205). In certain embodiments, a refrigerator unit or cryocooler is used in addition to or instead of the ambient air. When the refrigerator unit or cryocooler is used in addition to the ambient air, it may either cool the cooling unit (310) directly or pre-cool the ambient air prior to its passing over the cooling unit (310) and/or the canister, As illustrated in FIGS. 9A-9E, the cooling unit (310) comprises internal air passageways/ducts/funnels through which hot air exiting the CO₂ absorber (205) passes for cooling thereof, wherein the hot air enters the cooling unit (310) via inlets (311) and exits the cooling unit via a single outlet (312). While hot air passes in the ducts through the cooling unit (310), ambient air is pushed or blown, using any suitable means, such as fans (500, illustrated in FIGS. 4A-4B), across the external surface of the cooling unit (310), as well as across the external surface of the canister(s) (215). As noted above, each canister (215) comprises external ribs that increase external surface area, and as illustrated in FIG. 9C, the cooling unit (310) comprises ribs that increase external surface area, such that the blown air passes through such ribs, absorb heat, and thus cools the hot air within both the cooling unit (310) and the canister(s) (215). The ambient (or the pre-cooled) air may also pass between the passageways/ducts/funnels within the cooling unit (310) to improve heat removal.

In certain embodiments, the CO₂ absorber (205) is placed within a container (not shown) or underneath a heatshield cover (207) that prevents dispersion of air blown by, e.g., the fans (500), and directs it to the cooling unit (310) and the canisters (215) for cooling thereof. In addition, the container/cover further assists in separating the CO₂ absorber (205) from the counterlung/breathing bag (206) to prevent unintentional damage to the counterlung (206) due to excess heat generated in the CO₂ absorber (205).

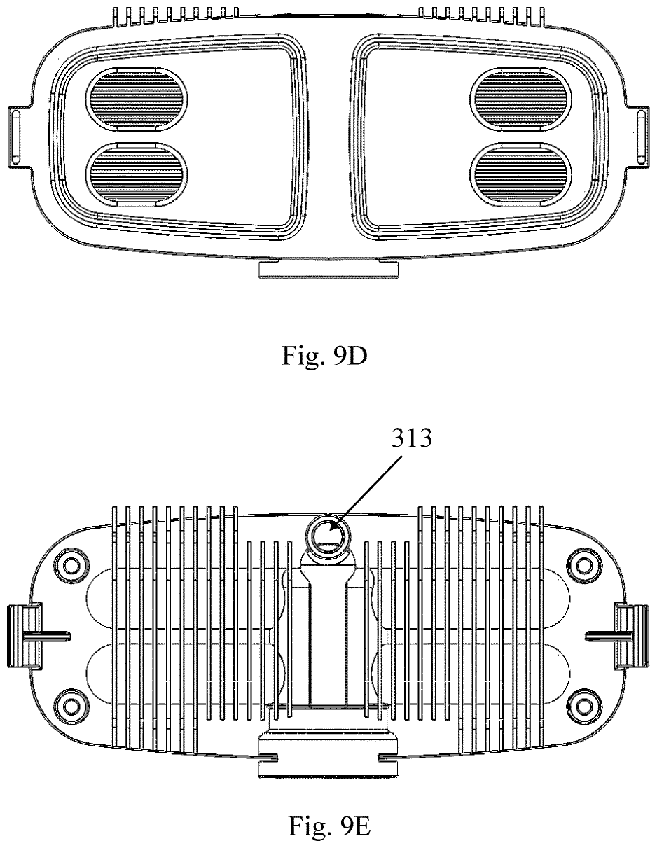

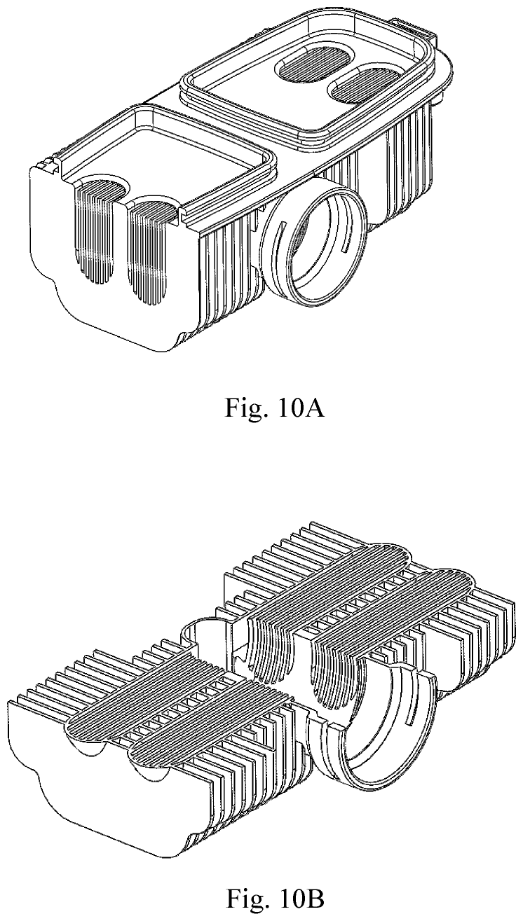

FIGS. 10A-10B are cross-sectional views of the cooling unit (310) of FIG. 9, showing the inner passageways/ducts/funnels through which the hot air exiting the CO₂ absorber (205) passes within the unit. As illustrated, the hot air exiting the CO₂ absorber (205) is divided into several passageways/ducts/funnels that constitute inner ribs to transfer “inner” heat outwardly to the outer ribs of the cooling unit (310). Notably, the cooling unit (310) can be fabricated in any suitable way. However, due to the fine structural architecture of the inner passageways/ducts/funnels, it is preferably manufactured by 3D-printing.

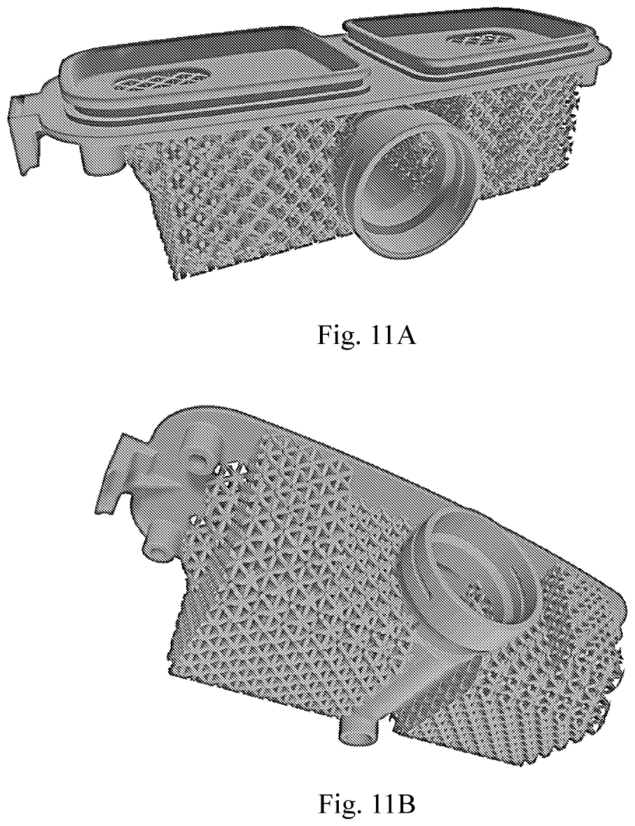

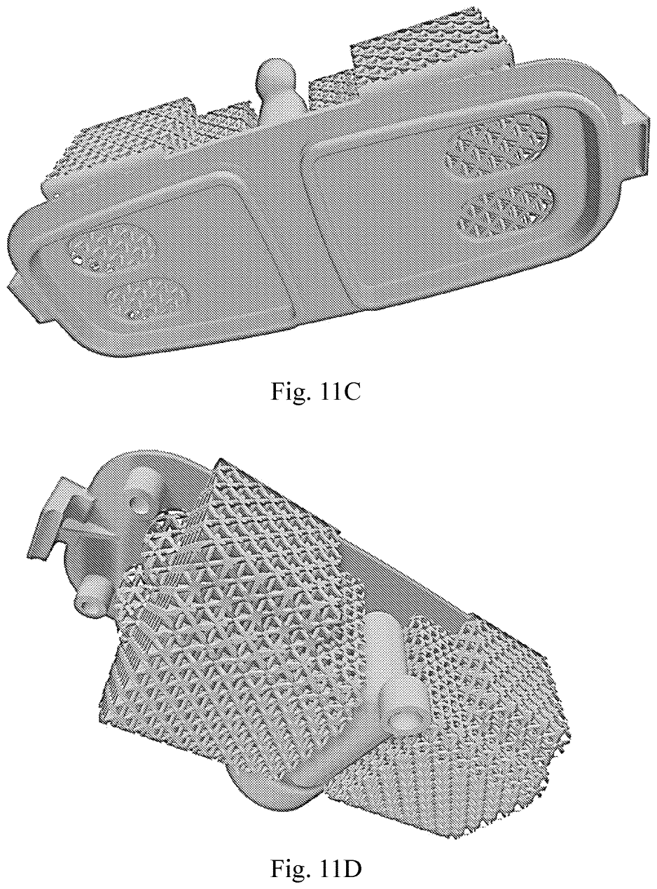

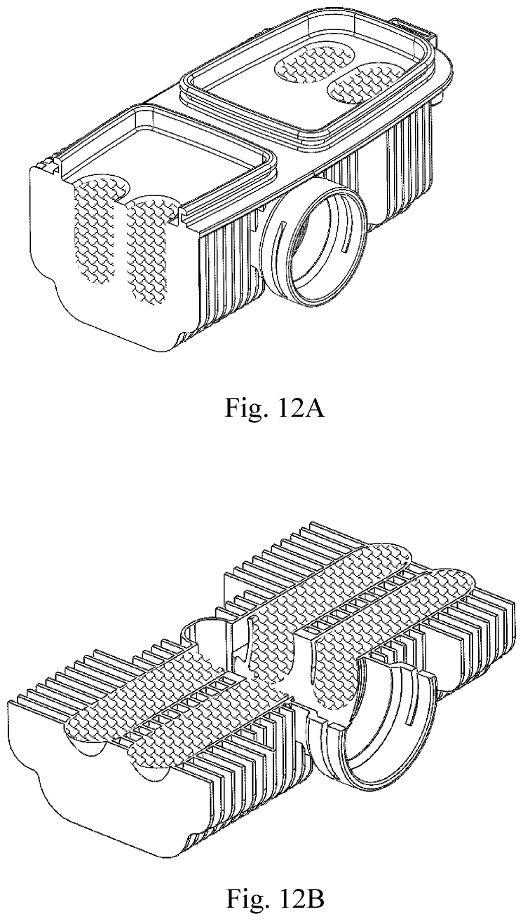

It should be noted that the cooling unit (310) can be formed in any suitable structure that allows heat dispersion. For instance, it may include ribs to increase its surface area as illustrated in FIGS. 9 and 10. Alternatively, or in addition, it may comprise a lattice structure-either only in the internal air passageways as illustrated in FIGS. 12A-12B, or also in the external perimeter as illustrated in FIGS. 11A-11D. Such lattice structure may further assist in strengthening the overall structure of the cooling unit (310) and/or aid in reducing its weight without affecting its cooling capabilities.

It is known that exhaled air contains moisture due to normal physiological processes, namely dry air that enters the lungs absorbs moisture from the lungs, mouth, and other tissues of the respiratory system. Accordingly, when the exhaled air is cooled by the cooling system (300), the moisture is condensed into liquid water. Such water may damage the CCR components and/or impair its activity, e.g., by clogging airways in the system.

Accordingly, as illustrated in FIGS. 9B and 9E, the cooling unit (310) includes a millibar discharge-valve (313) positioned at the bottom of the cooling unit (310) and designed to release condensed water into the environment. The release of the water is done automatically when enough water is accumulated without losing (a significant amount of) oxygen to the environment. Alternatively, the water release is done constantly without losing (a significant amount of) oxygen to the environment. Notably, the release of the water is carried out without enabling ambient air to enter the system.

In certain embodiments, the CCR (200) according to any of the embodiments above further comprises a computing system designed to constantly monitor in real time the temperature within the CCR, such as in the cooling system, in the counterlung (206), in the CO₂absorber (205), and just before air is delivered to the user's mouthpiece/gas breathing port). This is done using heat sensors/thermometers located at specific positions. The computing system then determines whether further cooling is required, and operates the fans (500) or cryocooler or both, according to need. For instance, when the user is under intense working conditions and breath heavily and fast, the cooling (i.e., fans' speed) is increased, whereas during rest or relaxed activity, the cooling is reduced. In further specific embodiments, the computing system communicates with a remote computing management system to externally monitor the user's condition.

1. A closed-circuit rebreather (CCR) (200) comprising:
a) a dismountable condensed oxygen cylinder (201) designed to provide oxygen;
b) an oxygen regulator (202) attached to said oxygen cylinder (201) and designed to provide oxygen at a predefined pressure;
c) a carbon dioxide (CO₂) absorber (205) comprising one or more dismountable canisters (215) designed to hold a CO₂ scrubber material;
d) a counterlung (206) connected to said CO₂ absorber (205) and designed to receive air exiting therefrom; and
e) a mouthpiece connected to said counterlung (206) via an inlet tube (203) and to said CO₂ absorber (205) via an outlet tube (204),
wherein said CCR further comprises a cooling system (300) that comprises a cooling unit (310) comprising: (i) hot air inlets (311) connected to an exit of said CO₂absorber (205) and designed to receive hot air passing through said CO₂ absorber (205); (ii) cold air outlets (312) designed to pass cold air to said counterlung (206); and a millibar discharge-valve (313) at its bottom section designed to discharge liquid water outside the CCR.

2. The CCR (200) of claim 1, wherein said cooling system (300) further comprises one or more fans or blowers designed to create airflow over said cooling unit (310) for cooling within the cooling unit (310) the hot air received at the hot air inlets (311).

3. The CCR (200) of claim 1, wherein said cooling system (300) further comprises a liquid-based cooling cycle holding a liquid coolant, said cooling cycle comprises an evaporator and a condenser, and optionally a pressure-or vacuum-generator.

4. The CCR (200) of claim 1, wherein said cooling system (300) further comprises an electric cryocooler designed to cool the air passing through the cooling unit (310).

5. The CCR (200) of claim 1, wherein said cooling system (300) further comprises: (i) one or more fans or blowers designed to create airflow over said cooling unit (310) for cooling the hot air passing therethrough; and (ii) a liquid-based cooling cycle or an electric cryocooler.

6. The CCR (200) of claim 1, further comprises a heatshield cover (207) positioned between said counterlung (206) and said CO₂absorber (205).

7. The CCR (200) of claim 1, further comprising a container holding said CO₂ absorber (205), and wherein said cooling system (300) is designed to direct airflow from said one or more fans or blowers over said cooling unit (310) and over said CO₂ absorber (205).

8. The CCR (200) of claim 1, wherein said CO₂absorber (205) comprises two or more dismountable canisters (215) designed to hold a CO₂ scrubber material.

9. The CCR (200) of claim 1, wherein said CO₂absorber (205) comprises an upper lid with a plunger (225) designed to press the CO₂ scrubber material within each of said canister(s) (215) and always maintain it under constant pressure.

10. The CCR (200) of claim 1, wherein each one of said one or more canisters (215) contains CO₂ scrubber material.

11. The CCR (200) of claim 1, wherein said CO₂absorber (205) comprises a mixer for mixing the CO₂ scrubber material within each of said one or more canisters (215).

12. The CCR (200) of claim 11, wherein said mixer is a physical mixer or is air-based.

13. A closed-circuit rebreather (CCR) (200) comprising:
a) a dismountable condensed oxygen cylinder (201) designed to provide oxygen;
b) an oxygen regulator (202) attached to said oxygen cylinder (201) and designed to provide oxygen at a predefined pressure;
c) a carbon dioxide (CO₂) absorber (205) comprising one or more dismountable canisters (215) designed to hold a CO₂ scrubber material,
d) a cooling system (300) connected to an exit of said CO₂ absorber (205);
e) a counterlung (206) connected to an exit of said cooling system (300) and designed to receive air exiting therefrom;
f) a heatshield cover (207) positioned between said counterlung (206) and said CO₂ absorber (205); and
g) a mouthpiece connected to said counterlung (206) via an inlet tube (203) and to said CO₂ absorber (205) via an outlet tube (204);
wherein:
said cooling system (300) comprises: (i) a cooling unit (310) that comprises hot air inlets (311) connected to an exit of said CO₂ absorber (205) and designed to receive hot air passing through said CO₂ absorber (205); and cold air outlets (312) designed to pass cold air to said counterlung (206); and a millibar discharge-valve (313) at its bottom section designed to discharge liquid water outside the CCR, wherein said cooling system (300) is attached to said CO₂ absorber (205) for receiving hot air passing therethrough; and (ii) one or more fans or blowers; and
said heatshield cover (207) is designed to direct airflow from said one or more fans or blowers over said cooling unit (310) and over said CO₂ absorber (205).

14. The CCR (200) of claim 13, wherein said CO₂ absorber (205) comprises an upper lid with a plunger (225) designed to press the CO₂ scrubber material within each one of said two or more canisters (215) and maintain it under constant pressure at all times.

15. The CCR (200) of claim 13, wherein said CO₂ absorber (205) comprises a mixer for mixing the CO₂ scrubber material within each one of said one or more canisters (215).

16. The CCR (200) of claim 13, wherein each one of said two or more canisters (215) contain CO₂ scrubber material.

17. The CCR (200) of claim 1, wherein said mouthpiece comprises a liquid discharge-valve.

18. The CCR (200) of claim 1, wherein all components of the CCR are dismantlable via quick-release latches.

19. The CCR (200) of claim 1, wherein the entire CCR unit is dismantlable via quick-release latches from a backpack designed to hold the CCR.
